(19) United States
(12) Patent Application Publication
(10) Pub. No.: US 2024/0394591 A1
(43) Pub. Date: Nov. 28, 2024

(54) CLOSEST CORRELATION METHOD (CCM) FOR MULTICLASS CLASSIFICATION

(71) Applicant: KONINKLIJKE PHILIPS N.V., EINDHOVEN (NL)

(72) Inventors: Yu-He Zhang, Cambridge, MA (US); Richard Earl Gregg, Cambridge, MA (US); Junmo An, Cambridge, MA (US)

(21) Appl. No.: 18/422,548

(22) Filed: Jan. 25, 2024

Related U.S. Application Data
(60) Provisional application No. 63/468,831, filed on May 25, 2023.

(51) Int. Cl. G06N 20/00; G16H 50/70

(52) U.S. Cl. CPC G06N 20/00; G16H 50/70

(57) ABSTRACT

A classifier visualization method includes determining an ordering of N classes that maximizes a gravity metric for the ordering computed as a sum of pairwise terms as a fraction with an accepted correlation coefficient of a corresponding pair of classes of the N classes in the numerator and a distance metric in the denominator that is indicative of a distance in the ordering between the classes; and displaying a confusion matrix for a classifier to be visualized, the displayed confusion matrix having the N classes ordered in the determined ordering along an X-axis and having the N classes ordered in the determined ordering along a Y-axis, and the value of each cell of the displayed confusion matrix corresponding to match counts between the class along the X-axis class at which the cell is located and the class along the Y-axis at which the cell is located.

### FIELD OF THE DISCLOSURE

The disclosed subject matter generally pertains to analysis of the performance of classifiers, utilizing a closest correlation method (CCM) to improve the performance and usability of confusion matrix for assessing classifier performance for correlated classes, and to medical image classifiers and computer aided diagnostic (CADx) systems and the like.

### BACKGROUND

Classifiers are used for a wide range of tasks, such as classification of photographs or images based on the depicted content, semantic classification of text-based documents, and so forth. In constructing a classifier, machine learning (ML) techniques are commonly used. In the medical imaging or radiology field, medical image classifiers are trained to perform various functionality such as computer aided diagnostic (CADx) analyses to detect indications of clinical findings (e.g., diseases, pathologies, or the like) in medical images acquired by magnetic resonance (MR) imaging, computed tomography (CT) imaging, positron emission tomography (PET) imaging, and other medical imaging modalities.

The performance of a classifier relates to how well the classifier output for a given input (e.g., input image) matches the correct or ground truth class. For example, if an image actually depicts a human (ground truth class), then an image classifier should correctly classify the image as depicting a human (classifier output). This is a correct classifier output. On the other hand, if the image classifier misclassifies the image as depicting a chimpanzee, this is an incorrect classifier output. In practice, some pairs of classes are more closely correlated (at the ground truth level) than others. For example, in a ground truth (e.g., anatomical similarity) sense, a human and a chimpanzee are more closely correlated than a human and a dolphin. Classifiers are more likely to make misclassifications between closely correlated classes (e.g., misclassifying a human as a chimpanzee) than between less correlated classes (e.g., misclassifying a human as a dolphin).

In analyzing performance of a classifier, it is useful to recognize the types of misclassifications the classifier is prone to making. This can be challenging in the case of a classifier (or group of classifiers) operating to classify items within a classification scheme having dozens, hundreds, or more classes, and in the case of classification relying on features that may not be readily recognizable. For example, a medical image classifier for CADx system may operate on image features derived from the medical image by image processing or analysis performed before applying the classifier. Such image features may include, for example, detected edges and characteristics of the edges (e.g., gradient magnitudes), corner features, blob features, ridge features, and/or so forth. To assess the types of misclassifications a classifier is prone to make, a confusion matrix is widely used in machine learning classification for visualization of the performance of an algorithm (i.e., classifier). The confusion matrix is a square matrix with the ground truth classes in a specified class order along one side (e.g., vertical or rows), and the classifier output in the same specified class order along the other side (e.g., horizontal or columns). In this arrangement, diagonal matrix elements of the confusion matrix correspond to the class output by the classifier being the same as the ground truth class (correct classification), while all off-diagonal matrix elements correspond to the class output by the classifier being different than the ground truth class (misclassification). For an ideal classifier that makes no misclassifications, all the diagonal elements of the confusion matrix will be 1 (assuming normalized values) indicating perfectly accurate classification, and every off-diagonal element will be zero indicating no instances of misclassification. Most real classifiers will not be ideal, and the confusion matrix for a nonideal classifier will have at least some diagonal elements with values of less than 1 (indicating some instances of the ground truth class being misclassified), and at least some off-diagonal elements with values greater than zero (these are the misclassifications).

It is desirable to provide a system with a graphical user interface (GUI) for calculating and presenting a confusion matrix, in which the confusion matrix is easily interpreted by a human viewer. For example, the elements of the confusion matrix can be color coded or shaded to emphasize elements with higher or lower values. However, a problem with effective presentation arises with the off-diagonal matrix elements representing the misclassifications. While the diagonal matrix elements correspond to correct classification, and are thus readily visually recognizable, the off-diagonal elements are typically not organized in any particular way. This can make it difficult for the human viewer to recognize the significance of misclassifications. For the previous example, if the off-diagonal matrix element for human-chimpanzee misclassifications has a high value, this may not be of too much concern since these classes are highly correlated (in the anatomical sense), so that a relatively high number of misclassifications between these classes may be expected. However, if the off-diagonal matrix element for human-dolphin misclassifications has a high value, this can indicate a significant problem with the classifier, since humans and dolphins have a low correlation (in the anatomical sense), so that a relatively high number of misclassifications between humans and dolphins is unexpected and is something that should be corrected by improved design of the classifier.

Some improvements are disclosed herein.

### BRIEF SUMMARY OF THE DISCLOSURE

According to one embodiment of the present disclosure, a system is disclosed that utilizes a closest correlation method (CCM) to improve the performance and usability of confusion matrix for correlated classes.

According to another embodiment of the present disclosure, a non-transitory computer readable medium stores instructions readable and executable by an electronic processor to perform a classifier visualization method including receiving or determining an accepted correlation coefficient between each pair of classes of N classes of classes of N classes, wherein N is equal to or greater than four; determining an ordering of the N classes that maximizes a gravity metric for the ordering, wherein the gravity metric is computed as a sum of pairwise terms in which each pairwise term comprises a fraction with the accepted correlation coefficient of a corresponding pair of classes of the N classes in the numerator and a distance metric in the denominator that is indicative of a distance in the ordering between the classes of the corresponding pair of the classes; and displaying a confusion matrix for a classifier to be visualized, the displayed confusion matrix having the N classes ordered in the determined ordering along an X-axis of the confusion matrix and having the N classes ordered in the determined ordering along a Y-axis of the confusion matrix, and the value of each cell of the displayed confusion matrix corresponding to match counts between the class along the X-axis class at which the cell is located and the class along the Y-axis at which the cell is located.

According to another embodiment of the present disclosure, a classifier visualization method includes receiving or determining an accepted correlation coefficient between each pair of classes of N classes of classes of N classes, wherein N is equal to or greater than four, wherein the N classes are N clinical findings, and a classifier to be visualized is a classifier configured to classify whether each of the N clinical findings is present in an input medical image; determining an ordering of the N classes that maximizes a gravity metric for the ordering, wherein the gravity metric is computed as a sum of pairwise terms in which each pairwise term comprises a fraction with the accepted correlation coefficient of a corresponding pair of classes of the N classes in the numerator and a distance metric in the denominator that is indicative of a distance in the ordering between the classes of the corresponding pair of the classes; processing each medical image of a plurality of medical images using the classifier to be visualized to determine, for each image, which of the N clinical findings is present in the medical image; determining classifier-computed match counts based on rates of co-occurrences of pairs of clinical findings in the output of the processing; and displaying a confusion matrix for a classifier to be visualized, the displayed confusion matrix having the N classes ordered in the determined ordering along an X-axis of the confusion matrix and having the N classes ordered in the determined ordering along a Y-axis of the confusion matrix, and the value of each cell of the displayed confusion matrix corresponding to the match counts between the class along the X-axis class at which the cell is located and the class along the Y-axis at which the cell is located.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 illustrates an example correlation coefficient matrix for unrelated classes, Human, Bird, Fish, Insect.

FIGS. 2a and 2b illustrate a confusion matrix heatmap for the 4 unrelated classes for a good algorithm (2a) and a bad algorithm (2b).

FIG. 3 illustrates a correlation coefficient matrix heatmap between Human, Chimp, Monkey, and Sloth.

FIG. 4 illustrates a correlation coefficient matrix heatmap with correlated classes arranged close together.

FIGS. 5a and 5b illustrate a confusion matrix heatmap arranged as disclosed herein for a good algorithm (5a) and a bad algorithm (5b).

FIGS. 6a and 6b illustrate a confusion matrix heatmap with random class arrangement for a good algorithm (6a) and a bad algorithm (6b).

FIG. 7 illustrates an example flowchart for the process to build CCCM.

FIGS. 8a and 8b illustrate a correlation coefficient matrix arranged by Hyper-G algorithm (8a) and its corresponding confusion matrix heatmap resulted by a perfect classifier (8b).

FIGS. 9a and 9b illustrate a correlation coefficient matrix with ECG labels arranged in alphabetical order (9a) and its corresponding confusion matrix heatmap resulted by a perfect classifier (9b).

FIGS. 10a and 10b illustrate a correlation coefficient matrix with ECG feature labels arranged by RTC algorithm (10a) and its corresponding confusion matrix heatmap resulted by a perfect classifier (10b).

FIG. 11 illustrates a classifier visualization system in accordance with the present disclosure.

FIG. 12 illustrates a flowchart of operations of a classifier visualization method using the system of FIG. 11.

### DETAILED DESCRIPTION OF EMBODIMENTS

As noted above, confusion matrix is widely used in machine learning classification for visualization of the performance of an algorithm (i.e., classifier). The matrices represent counts of actual (i.e., ground truth) against predicted values (i.e., predicted by the classifier), with the row representing the actual classes and the column representing the predicted classes, or vice versa. The confusion matrix provides a clear visual representation when the classes are not correlated. If an algorithm performs with 100% perfection, when the actual values always agree with the prediction, the count values or 100% only appear in the diagonal elements and all other elements are zeros or 0%. The worse the algorithm performance, the more non-diagonal elements will present higher counts or percentages. The count values and percentages are often color-coded like a heat map so that a quick visual assessment can be performed.

When some of the classes are correlated, the presentation of the confusion matrix becomes cluttered even if an algorithm performs with perfect predictions. In this case, while the diagonal counts still represent the agreement between the actual and predicted values, the off-diagonal counts no longer necessarily represent the false outcome of the algorithm. This makes the visual assessment more difficult, often inconclusive, and severely limits the use of the confusion matrix for broader classifications.

Since correlated classes are common and often need to be evaluated together, improvement is highly desirable so that the confusion matrix can continue to be effectively used. This application provides such a method to ensure the desired counts appear in the close vicinity of the diagonal elements.

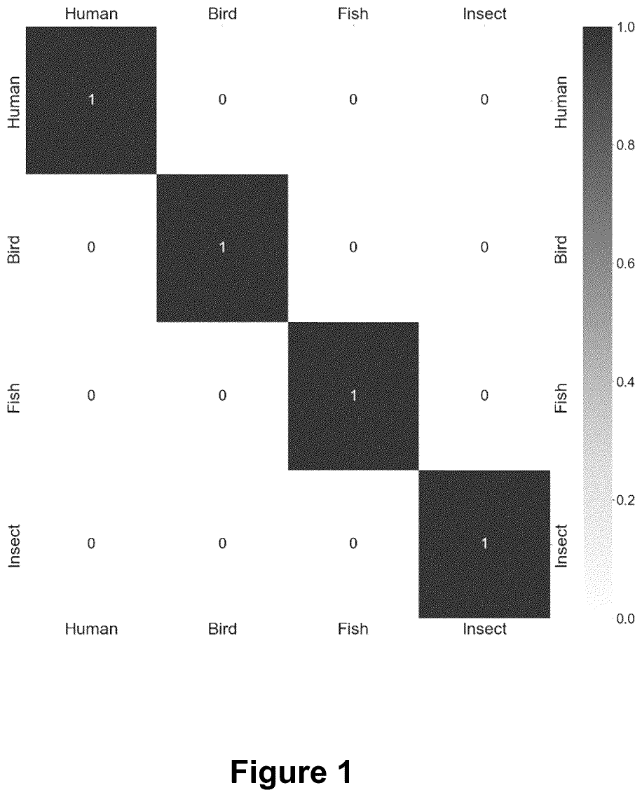

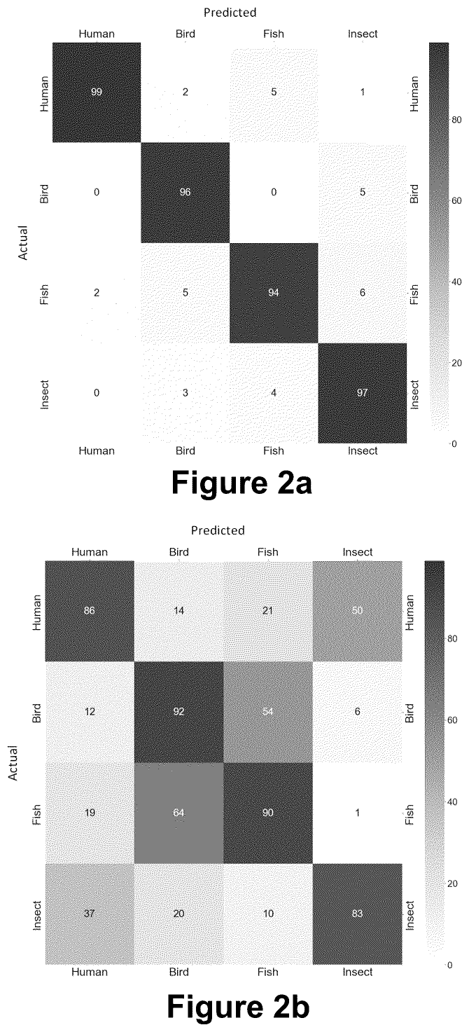

First, take an example for a classification scheme including four unrelated animals ordered as follows: [‘Human’, ‘Bird’, ‘Fish’, ‘Insect’]. The self-correlation-coefficients are 1. The correlation coefficients between any of the four animals are 0. The correlation coefficient matrix is shown in FIG. 1. When an algorithm is evaluated to classify these 4 animals, any off-diagonal element with non-zero values (darker colored) will indicate misclassification. Since correlation coefficients for all off-diagonal elements are equally zeros, it does not matter where the off-diagonal dark element appears. FIG. 2a shows the confusion matrix for a good algorithm, and FIG. 2b shows the confusion matrix for a bad algorithm.

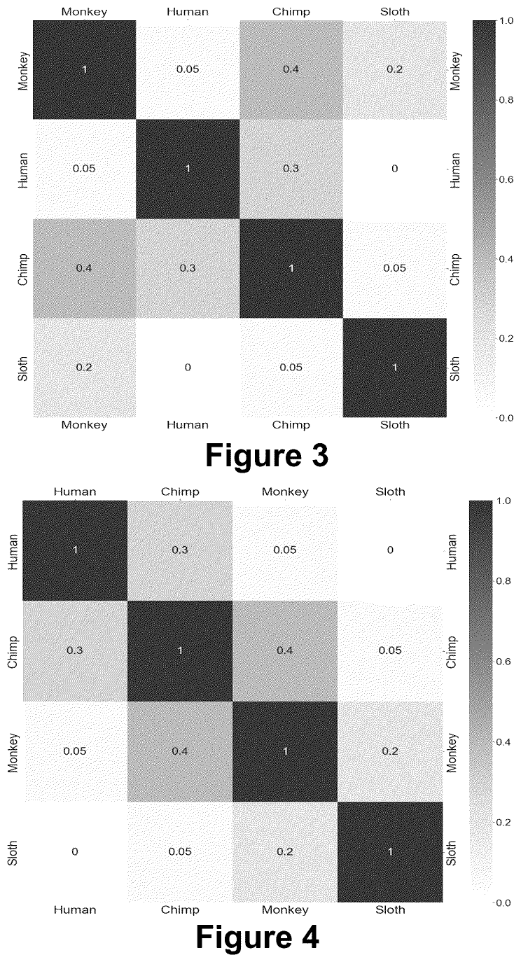

Now, take an example for a list of 4 correlated mammals, [‘Human’, ‘Chimp’, ‘Monkey’, ‘Sloth’]. Correlation coefficients are used to represent how strongly one class is correlated to the other class, with 1 being fully correlated and 0 being no correlation. The correlation coefficients between classes can be generated in different ways, and in general the correlation coefficient between a pair of classes can be viewed as representing the actual correlation or actual similarity between those classes. In the previous example, the correlation coefficients could be generated based on anatomical similarity, so that the pair (human, chimpanzee) would have a higher correlation coefficient than the pair (human, dolphin). In a CADx context, a class pair such as (complete fracture, partial fracture) might have a high correlation in an imaging modality in which both types of fracture show up in a CT image as a high contrast line crossing a bone; whereas, a class pair such as (linear fracture, transverse fracture) may have a low correlation coefficient since the linear fracture shows up as a high contrast line about parallel with the shaft of the bone while the transverse fracture shows up as a high contrast line about perpendicular the shaft of the bone. These are merely nonlimiting illustrative examples. For the simplicity of illustration, the correlation coefficients have already been created. Human and Chimp are relatively closely related, with a correlation coefficient of 0.3. Chimp and Monkey are closer with a correlation coefficient of 0.4. Human and Sloth are very unrelated with a correlation coefficient of 0. And so on. The correlations can be illustrated in a correlation coefficient matrix as shown in FIG. 3. Optionally, the correlation coefficients may be color-coded to present the confusion matrix as a heatmap, for example using darker color for higher correlated elements and lighter color for lower correlated elements. A confusion matrix from testing results by a perfect algorithm in classifying between Human, Chimp, Monkey, and Sloth is expected to have the exact same pattern as this pattern of correlation coefficient matrix. In FIG. 3, the diagonal elements are self-correlated therefore appear darkest. But the matrix in does not have a clear pattern for the off-diagonal elements and is not suitable to use for visual evaluation. It will be even more confusing if the list of classes is longer with a much bigger matrix.

In embodiments disclosed herein, the order of the classes is arranged (as the labels of the horizontal and vertical axis) for the matrix based on their correlation levels, the higher the correlation the closer they are arranged, so that a clear pattern is formed. FIG. 4 shows the result by re-arrangement of classes.

With the clear and meaningful pattern shown in FIG. 4, the visual evaluation becomes much more relevant and straightforward. The classification algorithm will not be viewed as robust if high counts occur at the elements further away from the diagonal. The algorithm will be viewed as poorly performed if high counts occur at the corners that are furthest away from the diagonal, i.e., a sloth is frequently classified as a human, or vice versa.

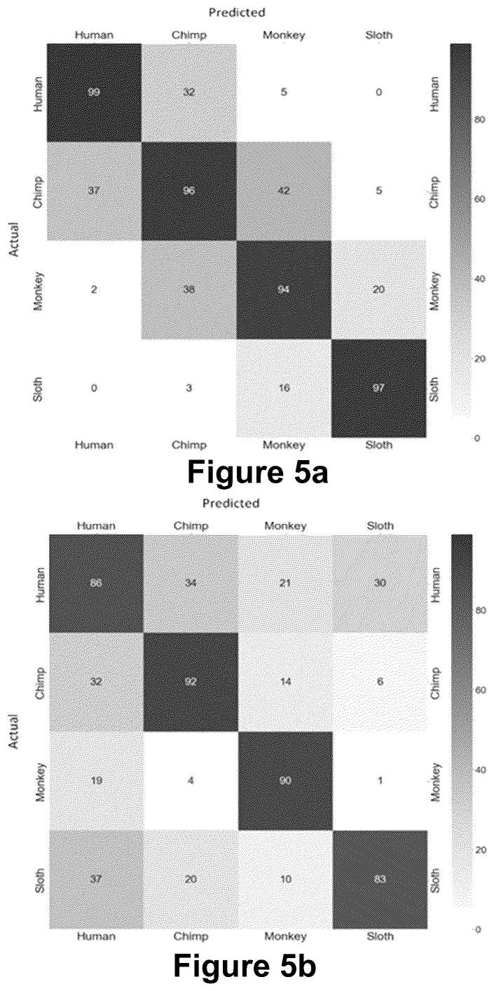

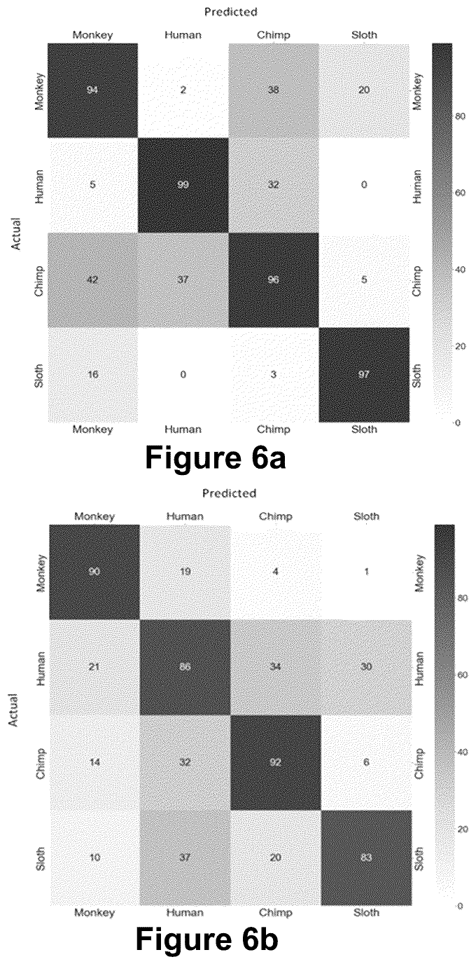

Now let's take a look at how one can use the proposed idea to evaluate two algorithms, one good and the other bad. FIGS. 5a and 5b shows the confusion matrices of the testing results from the good algorithm (FIG. 5a) and the bad algorithm (FIG. 5b) with the classes arranged (or ordered) using the approach disclosed herein. Note the element values for the confusion matrix are integers indicating how many instances of an actual class are predicted by the algorithm. With the arrangement as disclosed herein, the pattern of the good algorithm is well organized with high values distributed on or close to the diagonal while the one with the bad algorithm is not. As a comparison, FIGS. 6a and 6b show the confusion matrices of the testing results from the good algorithm (FIG. 6a) and the bad algorithm (FIG. 6b) with the classes arranged randomly. One cannot tell the good algorithm from the bad based on the patterns since both look random.

Correlated classes are not suitable for existing confusion matrix usage. True positive match can appear in elements far away from the diagonal so that high counts in off-diagonal elements cannot be used as an indication of algorithm deficiency. In the field of machine learning, Python data analysis libraries such as SciPy, Pandas, Scikit-learn, Seaborn, and NumPy are commonly used for statistical evaluations and visualization. These libraries provided APIs for correlation and confusion matrices. But none of them offers a good method for visualizing the performance of a classification algorithm on correlated classes with desired clarity. The standard method for a multilabel problem (labels are not mutually exclusive) is a single confusion matrix for each class as a 2×2 matrix, that class versus all other classes lumped together. Mistakes are known by off-diagonal position, but the kind of misclassification is unknown.

Since the algorithm disclosed herein is formulated such that the more correlated classes are arranged close together and therefore off-diagonal elements of correlated classes are close to the matrix diagonal while classes that are less correlated are far from the diagonal. This arrangement helps the user understand which off-diagonal elements are likely to be misclassifications.

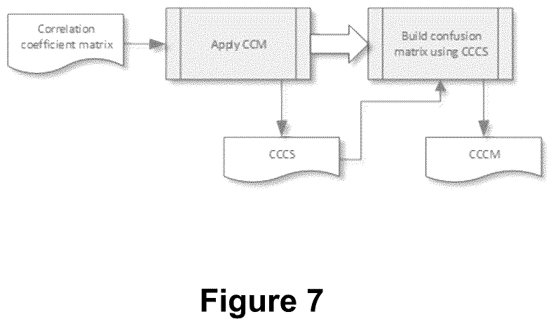

Embodiments herein utilize a closest correlation method (CCM) to improve the performance and usability of confusion matrix for correlated classes. The class elements are arranged such that the closely correlated classes are positioned close together. There are potentially different methods to achieve this optimized class arrangement. These methods can be called the Closest Correlation Methods (CCMs) in general. The sequence of the classes that is identified by the CCM is called the Closest Correlated Class Sequence (CCCS). The confusion matrix that is built upon the CCCS is called the Closest Correlated Confusion Matrix (CCCM). FIG. 7 is a flow chart to illustrate the process to build the CCCM. Basically, the process starts with a list of classes in an arbitrary initial order. The process uses some existing algorithm to find the correlation coefficients between each and every pair of the classes in the list. It then applies a CCM method to identify the CCCS. Finally, it uses CCCS as the horizontal and vertical axes to build the CCCM that will be used to assess the candidate classification algorithms.

The following are the process and main elements, with a novel CCM algorithm,

A correlation coefficients matrix is built as the starting point of the method. This process assumes the correlation coefficient values have already been identified. The steps up to Step 8 are explanation of the correlation coefficients instead of how they are derived.

The correlation coefficient is a statistical measure of the strength of a linear relationship between two classes. Its values range from −1 to 1. 1 indicates the perfect positive correlation, −1 indicates the perfect negative correlation or mutual exclusivity, and 0 indicates no linear relationship between them.

The value of a given element in the matrix is the correlation coefficient between the corresponding row class and the column class.

The diagonal elements are self-correlation coefficients and therefore valued at 1's.

The correlation coefficient for a pair of independent classes is 0.

The correlation coefficient for a pair of mutually exclusive classes is less than 0, with −1 for 100% exclusivity.

The correlation coefficient for correlated classes is greater than 0.

The above correlation coefficient method is based on existing algorithm and can be Pearson, Spearman, or Kendall.

An algorithm is invented and formulated such that the correlated classes are arranged close together and therefore off-diagonal elements of correlated classes are close to the matrix diagonal while classes that are less correlated are far from the diagonal.

The algorithm is called Hyper-G, which stands for maximum gravity.

The Hyper-G algorithm starts with a one-dimensional (1D) array of N class elements.

A variable r is defined as the distance between a pair of elements in the 1D array, with r=1 for the adjacent pair. The distance is increased by 1 for each additional element position.

The gravity between a pair of elements along the 1D class array is defined as g=c/r^{q}, where c is the correlation coefficient and r is the distance. The power q is a positive real number, with q=1 for regular 2D space. When q>1, the gravity converges faster with reduced distance, emphasizing the influence of the closest class elements.

The total gravity of the 1D class array is the summation of gravities between each and every possible pair of the class elements with the following formula,

$$G = {\sum }_{i = 0}^{N - 1} {\sum }_{j = i + 1}^{N - 1} {g}_{ij} = {\sum }_{i = 0}^{N - 1} {\sum }_{j = i + 1}^{N - 1} \frac{{c}_{ij}}{{r}_{{ij}^{q}}} \qquad Eq . [ 1 ]$$

The 1D class array is rearranged and a new total gravity is calculated.

The algorithm exhausts all possible permutations of the class element arrangement in the 1D array, and the total gravity is calculated for each and every permutation.

The permutation with the greatest total gravity is the final 1D class array arrangement choice that is used to form the Hyper-G confusion matrix.

As an alternative variation of Hyper-G algorithm, the gravity can be neglected after a certain distance, which is defined as Varnishing Distance denoted as Dᵥ. Dᵥ can be as near as 2 and as far as N, Dᵥ∈[2, N].

With Varnishing Distance, Eq. [1] can be reformulated as following,

$$G = {\sum }_{i = 0}^{N - 1} {\sum }_{j = i + 1}^{min ( i + {D}_{v} - 1 , N - 1 )} {g}_{ij} = {\sum }_{i = 0}^{N - 1} {\sum }_{j = i + 1}^{min ( i + {D}_{v} - 1 , N - 1 )} \frac{{c}_{ij}}{{r}_{{ij}^{q}}} \qquad Eq . [ 2 ]$$

where min (i+Dᵥ−1, N−1) is smaller of i+Dᵥ−1 and N−1.

As a comparison, a very simple method called Ranking Total Correlation (RTC) is used to put correlated classes close together without taking into account the influence of distance. It calculates the sum of all the correlation coefficients for each class. The class with the highest total correlation coefficients starts at the center of the 1D class array, and the other classes are arranged around the central class element in descending order of the total correlation coefficient. The following is the process to build Hyper-G confusion matrix,
1. For a given list of N classes, form a 1D array, denoted as

$$P = \{ {P}_{0} , {P}_{1} , … , {P}_{i} , … , {P}_{N - 1} \} ,$$

where Pᵢ is the ith class element in P.
2. The correlation coefficient between the jth and the ith class elements is denoted as c[Pᵢ, Pⱼ].
3. The distance between the jth and the ith elements is denoted as r[i,j]=j−i.
4. Calculate the total gravity of P with the formula defined in Eq. [1] or Eq. [2].
5. Rearrange the 1D class array and calculate the new total gravity.
6. Exhaust all possible permutations of the 1D class array arrangement and calculate the total gravity for each and every permutation.
7. The permutation with the greatest total gravity is the final class arrangement sequence for the Hyper-G confusion matrix.
8. The following pseudo code illustrate the above-described process,

```
q = 1;
Gₘₐₓ = 0;
Dᵥ = 3;
for (each P in All_Possible_Permutations( ))
        G = 0;
  for (i = 0; i < N; i + +)
    for (j = i + 1; j < min(i + Dᵥ, N); j + +)
      r[i, j] = j − i;
      $G = G + \frac{c [ {P}_{i} , {P}_{j} ]}{{( r [ i , j ] )}^{q}} ;$
  if (G > Gₘₐₓ)
    Gₘₐₓ = G;
    Pₘₐₓ = P;
```

The final Pₘₐₓ is used as the horizontal and vertical axes to form the Hyper-G confusion matrix.

Now for at an example with a list of ECG feature classes (labels) [‘AFIB’, ‘ISC’, ‘LAD’, ‘LAE’, ‘LAFB’, ‘LBBB’, ‘LVH’, ‘RAE’, ‘RBBB’, ‘RVH’, ‘RAE’, ‘LAE’, ‘LBBB’, ‘LVH’, ‘LAD’, ‘STT’, ‘ISC’, ‘AFIB’, ‘LAFB’, ‘RBBB’]. Table 1 listed these labels with corresponding features.

TABLE 1
ECG feature labels used in the examples.

| Label | ECG Feature |
|---|---|
| AFIB | Atrial fibrillation |
| ISC | Ischemia |
| LAD | Left axis deviation |
| LAE | Left atrial enlargement |
| LAFB | Left anterior fascicular block |
| LBBB | Left bundle branch block |
| LVH | Left ventricular hypertrophy |
| RAE | Right atrial enlargement |
| RBBB | Right bundle branch block |
| RVH | Right ventricular hypertrophy |
| STT | ST-T abnormality |

Table 2 shows the correlation coefficients between each of the ECG feature classes. Note that the feature labels are listed in alphabetical order.

TABLE 2
The correlation coefficients between a list of ECG feature classes.

| | AFI | | | | LAF | LBB | | | RBB | | |
|---|---|---|---|---|---|---|---|---|---|---|---|
| | B | ISC | LAD | LAE | B | B | LVH | RAE | B | RVH | STT |
| AFIB | 1.000 | 0.118 | 0.042 | −0.086 | 0.044 | 0.040 | 0.031 | −0.029 | 0.051 | 0.006 | 0.041 |
| ISC | 0.118 | 1.000 | 0.022 | 0.040 | 0.010 | −0.028 | 0.061 | −0.001 | 0.015 | 0.004 | 0.124 |
| LAD | 0.042 | 0.022 | 1.000 | 0.032 | 0.008 | 0.162 | 0.043 | −0.009 | 0.046 | −0.007 | 0.016 |
| LAE | −0.086 | 0.040 | 0.032 | 1.000 | 0.020 | 0.044 | 0.077 | −0.016 | 0.023 | 0.008 | 0.038 |
| LAF | 0.044 | 0.010 | 0.008 | 0.020 | 1.000 | 0.040 | 0.009 | −0.005 | 0.186 | −0.007 | 0.016 |
| B | | | | | | | | | | | |
| LBB | 0.040 | −0.028 | 0.162 | 0.044 | 0.040 | 1.000 | −0.033 | 0.006 | −0.046 | −0.012 | −0.022 |
| B | | | | | | | | | | | |
| LVH | 0.031 | 0.061 | 0.043 | 0.077 | 0.009 | −0.033 | 1.000 | 0.015 | −0.030 | −0.014 | 0.153 |
| RAE | −0.029 | −0.001 | −0.009 | −0.016 | −0.005 | −0.006 | 0.015 | 1.000 | −0.004 | 0.041 | 0.004 |
| RBB | 0.051 | 0.015 | 0.046 | 0.023 | 0.186 | −0.046 | −0.030 | −0.004 | 1.000 | 0.042 | −0.024 |
| B | | | | | | | | | | | |
| RVH | 0.006 | 0.004 | −0.007 | 0.008 | −0.007 | −0.012 | −0.014 | 0.041 | 0.042 | 1.000 | 0.022 |
| STT | 0.041 | 0.124 | 0.016 | 0.038 | 0.016 | −0.022 | 0.153 | 0.004 | −0.024 | 0.022 | 1.000 |

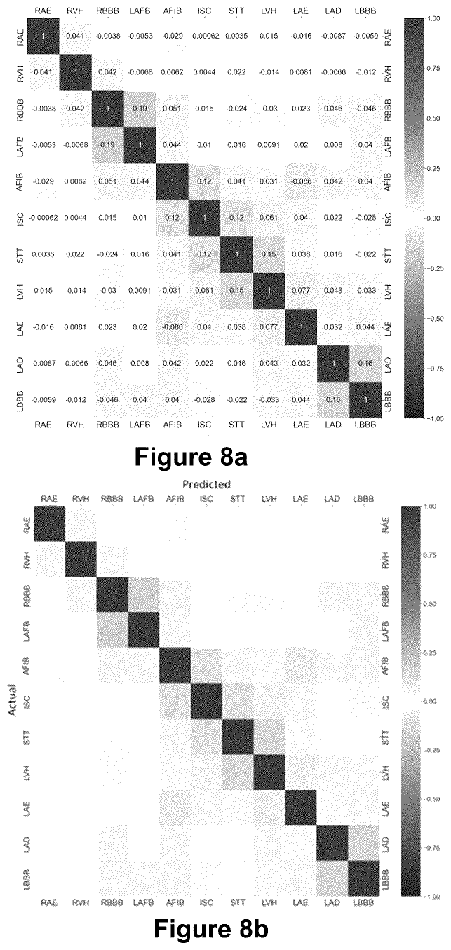

With correlation coefficients given in Table 2, by applying the Hyper-G algorithm a Hyper-G order of the ECG feature labels is obtained [‘RAE’, ‘RVH’, ‘RBBB’, ‘LAFB’, ‘AFIB’, ‘ISC’, ‘STT’, ‘LVH’, ‘LAE’, ‘LAD’, ‘LBBB’], and a correlation coefficient matrix that can be illustrated in FIG. 8a. With the Hyper-G order of feature labels, the expected heatmap for the confusion matrix of a perfect classifier is shown in FIG. 8b. Optionally, the heatmap may be color-coded such that the elements between positively correlated classes are in warm color, the elements between negatively correlated classes are in cold color, and the elements between independent classes are in white color. The intensity of the color in such a heat map increases with the correlation. When a new classifier is tested, a confusion matrix not following the Hyper-G heatmap will indicate an algorithm degradation. The darker elements appear away from the diagonal the worse the classifier.

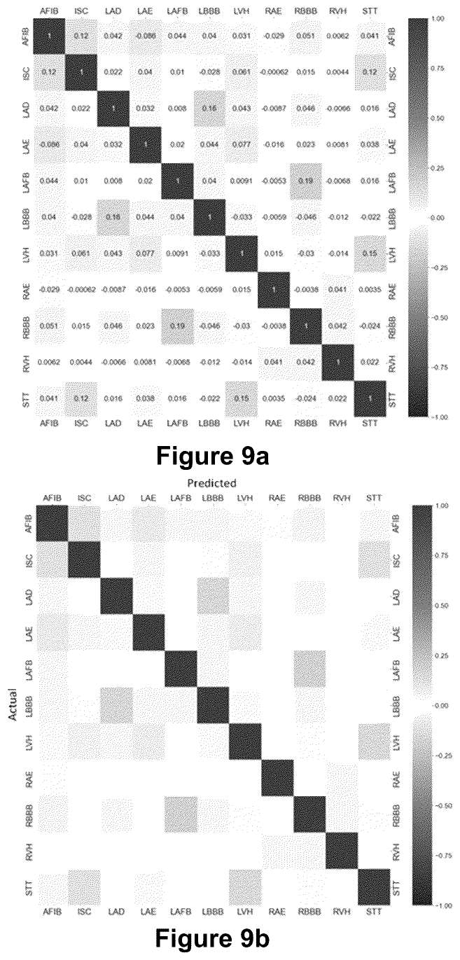

To further illustrate the benefit of the Hyper-G algorithm, FIGS. 9a and 9b show the correlation coefficient matrix with ECG feature labels arranged in alphabetical order (FIG. 9a) and its corresponding confusion matrix heatmap resulted by a perfect classifier (FIG. 9b). Since the dark elements spread over the heatmap including the elements furthest away from the diagonal, there is not a clear pattern to expect for a good classifier. A bad classifier would look similar.

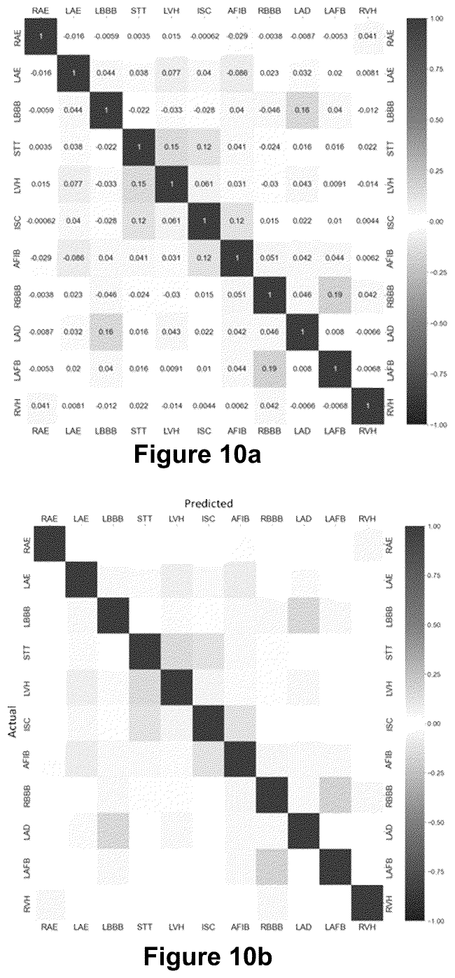

As mentioned previously, RTC is a simple and faster algorithm to find a better order of the classes than a random order. FIGS. 10a and 10b show the correlation coefficient matrix with ECG labels arranged by RTC algorithm (FIG. 10a) and its corresponding confusion matrix heatmap resulted by a perfect classifier (FIG. 10b). The heatmap pattern does show some improvement from the one that arranged by alphabetical order. But one can see that the heatmap pattern is not optimized. The clarity in the heatmap pattern by Hyper-G is much higher.

The correlation coefficient values used in the examples are based on clinical data. For instance, left anterior fascicular block (LAFB) and right bundle branch (RBBB) occur commonly and also occur together commonly, therefore they have a higher correlation than other labels. Similarly, left atrial enlargement (LAE) and left ventricular hypertrophy (LVH) occur together commonly because they are related clinically. When the left ventricle has difficulty pumping blood into the body, it becomes muscular, i.e., hypertrophied. When the left ventricle is hypertrophied, it becomes hard for the left atrium to pump into the left ventricle, therefore the left atrium also becomes hypertrophied (LAE). A similar phenomenon happens with the right ventricle and right atrium except the right ventricle pumps into the lungs. It is also known that pulmonary issues lead to right ventricular hypertrophy (RVH) which then leads to right atrial enlargement (RAE), therefore RVH and RAE occur together as labels.

Hyper-G confusion matrix shown in FIG. 8 demonstrated a clear advantage over other arrangements, such as alphabetical or random order and the RTC algorithm. High counts in or close to diagonal elements and low counts in elements aways from diagonal indicate a good classification algorithm. In contrast, high counts occurred in elements further away from diagonal indicate a poor performance from the algorithm.

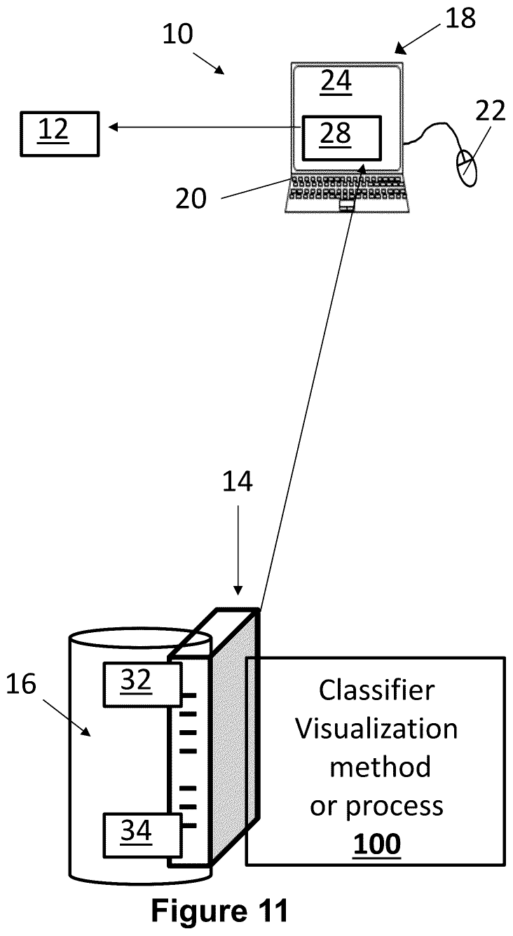

With reference to FIG. 11, an illustrative apparatus 10 for generating a confusion matrix 12 for a classifier 34 to be visualized is shown. As shown in FIG. 11, the apparatus 10 includes, or is accessible by, a server computer 14. The server computer 14 comprises a computer or other programmable electronic device that includes or has operative access to a non-transitory computer readable medium. It will be appreciated that the server computer 14 could be implemented as a plurality of server computers, e.g., interconnected to form a server cluster, cloud computing resource, or so forth, to perform more complex computational tasks. The server computer 14 can comprise one or more non-transitory storage media 16.

An electronic processing device 18, such as a workstation computer, or more generally a computer, a mobile device (e.g., a tablet computer), is operable by a service engineer (SE), information technology (IT) professional, or the like to provide a user interface with a classifier visualization method or process 100 running on the server computer 14.

The electronic processing device 18 includes typical components for a user interfacing computer, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18, or may include two or more display devices. To display a heat map the display device 24 should be a color display device.

The non-transitory storage media 16 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 16 herein is to be broadly construed as encompassing a single medium or multiple media of the same or different types. Likewise, the electronic processor 20 may be embodied as a single electronic processor or as two or more electronic processors. The non-transitory storage media 16 stores instructions executable by the at least one electronic processor 20. The instructions include instructions to generate a visualization of a graphical user interface (GUI) 28 for display on the display device 24.

The electronic processing device 18 can communicate with the server computer 14 via a communication link, which typically comprises an electronic network including the Internet augmented by local area networks (e.g. LAN or WAN) for electronic data communications. The electronic processing device 18 may be a dumb terminal connected with the server computer 14 via a LAN, WAN, Internet, or so forth. In the illustrative example, the server computer 14 handles the computation of the confusion matrix 12 as disclosed herein, and the electronic processing device 18 primarily serves as the user interfacing device including displaying the confusion matrix 12 on the display 24. However, this is merely an illustrative example, and the processing may be variously distributed between the server 14 and user interfacing processing device 18. In some embodiments, only one computer may be provided which includes all functionality—e.g., the server computer 14 could be omitted and its functionality performed solely by the single user interfacing computer 18.

The apparatus 10 is configured as described above to perform a classifier visualization method or process 100. The non-transitory storage media 16 stores instructions executable by the server computer 14 (and/or by the electronic processing device 18) to perform the classifier visualization method or process 100 which includes presenting a heat map 32 representing the confusion matrix 12 for a classifier 34 to be visualized. In some examples, the method 100 may be performed at least in part by cloud processing (that is, the server computer 14 may be implemented as a cloud computing resource comprising an ad hoc network of server computers).

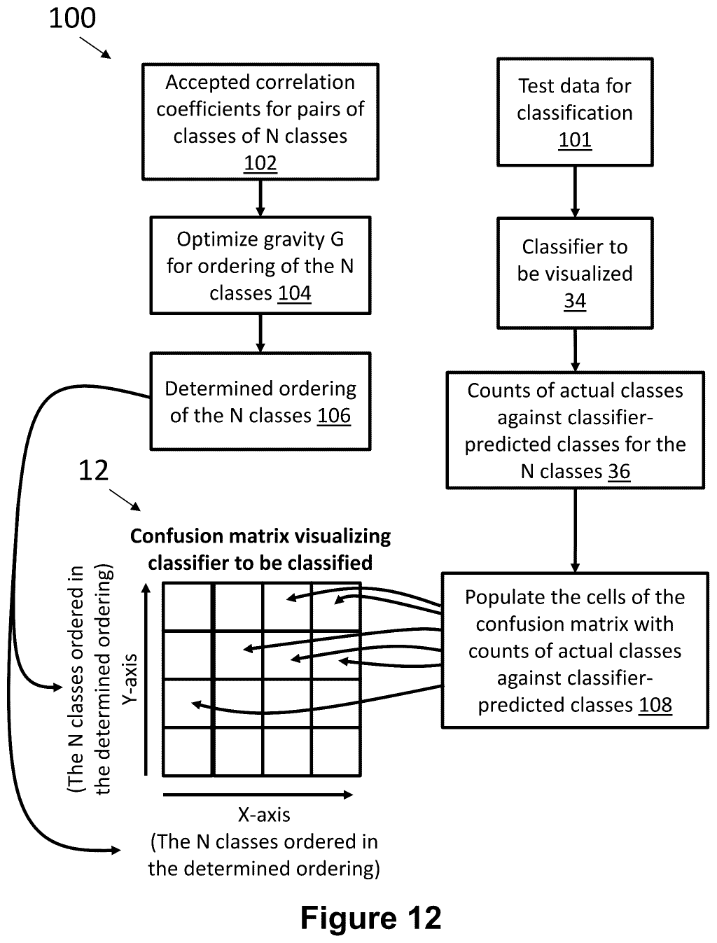

With reference to FIG. 12, and with continuing reference to FIG. 11, an illustrative embodiment of an instance of the classifier visualization method 100 is diagrammatically shown as a flowchart.

The method 100 uses test data 101 to be classified. For example, if the classifier 34 is a medical images classifier for a computer aided diagnosis (CADx) system that classifies images as to whether they depict features of various clinical findings, then the test data 101 may be a set of labeled medical images, in which each image is labeled as to the ground truth class. (For example, if the medical image shows a clinical finding of a compound fracture, then that image is suitably labeled with the ground truth class “compound fracture” by a radiologist or other qualified diagnostician). At an operation 102, an accepted correlation coefficient between each pair of classes of N classes is received, or determined, by the server computer 14. As previously mentioned, this could be done in various ways, such as by assessment of a radiologist or other qualified diagnostician (or group of diagnosticians) based on their experience and based on anatomical similarities of different clinical findings (for the example of a CADx classifier). In another example, the operation 102 could be done in an automated fashion by analyzing radiology reports of actual patients. In such radiology reports, it is sometimes the clinical practice that any correction to the report be made by way of an addendum that is appended to the original radiology report. By analyzing such radiology reports, a closely correlated clinical finding pair (A, B) can be identified by the presence of a (relatively) high number of radiology reports presenting finding A in the original report and being corrected to finding B in the addendum. These are merely nonlimiting examples of approaches for implementing operation 102. In some embodiments, N is equal to or greater than four, and in many practical cases N will be much larger than four (e.g., N may be on the order of a dozen, a few dozen, or hundreds of classes). For the illustrative CADx classifier application, the N classes are N clinical findings, and the classifier 34 to be visualized is a classifier configured to classify whether each of the N clinical findings is present in an input medical image.

At an operation 104, an ordering of the N classes that maximizes a gravity metric for the ordering is determined. In some embodiments, the gravity metric is computed as a sum of pairwise terms in which each pairwise term comprises a fraction with the accepted correlation coefficient of a corresponding pair of classes of the N classes in the numerator and a distance metric in the denominator that is indicative of a distance in the ordering between the classes of the corresponding pair of the classes.

In one example embodiment, the gravity metric comprises:

$$G = {\sum }_{i = 0}^{N - 1} {\sum }_{j = i + 1}^{N - 1} \frac{{c}_{ij}}{{r}_{{ij}^{q}}}$$

where G is the gravity metric, and cᵢⱼ is the accepted correlation coefficient for the pair of classes i and j, and r_{i,j} is a distance metric indicative of a distance in the ordering between the classes i and j, and q is a positive real value.

In another example embodiment, the gravity metric comprises:

$$G = {\sum }_{i = 0}^{N - 1} ( {\sum }_{j = i + 1}^{min ( i + {D}_{v} - 1 , N - 1 )} \frac{{c}_{ij}}{{r}_{{ij}^{q}}} )$$

where G is the gravity metric, and cᵢⱼ is the accepted correlation coefficient for the pair of classes i and j, and rᵢⱼ is a distance metric indicative of a distance in the ordering between the classes i and j, and q is a positive real value, and Dᵥ is a vanishing distance having a value between 2 and N.

In either of these example embodiments, q can be either equal to 1 or greater than 1.

In other embodiments, the determining of the ordering of the N classes that maximizes the gravity metric for the ordering includes computing the gravity metric for each of the N! orderings of the N classes, and selecting the determined ordering as the ordering of the N classes for which the computed gravity metric is largest.

At an operation 106, the confusion matrix 12 for the classifier 34 to be visualized is displayed on the display device 24. The classifier 34 to be visualized comprises a single N-class classifier or N single-class classifiers (again, where N is greater than or equal to four). As shown in FIG. 12, the displayed confusion matrix 12 has the N classes ordered in the determined ordering along an X-axis of the confusion matrix 12 and having the N classes ordered in the determined ordering along a Y-axis of the confusion matrix 12. The value of each cell of the displayed confusion matrix 12 corresponding to match counts 36 between the class along the X-axis class at which the cell is located and the class along the Y-axis at which the cell is located.

In some embodiments, the match counts 36 between each pair of classes of the N classes can be computed by processing each medical image of a plurality of medical images using the classifier 34 to be visualized to determine, for each image, which of the N clinical findings is present in the medical image, and determining classifier-computed match counts based on rates of co-occurrences of pairs of clinical findings in the output of the processing.

In some embodiments, the displayed confusion matrix 12 comprises a heat map in which the value of each cell of the displayed confusion matrix corresponding to the match counts 36 between the class along the X-axis class at which the cell is located and the class along the Y-axis at which the cell is located is represented as a color.

Once the confusion matrix 12 is generated, then at an operation 108, the cells of the confusion matrix 12 can be populated with counts of actual classes against classifier-predicted classes.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A non-transitory computer readable medium storing instructions readable and executable by an electronic processor to perform a classifier visualization method, the classifier visualization method comprising:
receiving or determining an accepted correlation coefficient between each pair of classes of N classes of classes of N classes, wherein N is equal to or greater than four;
determining an ordering of the N classes that maximizes a gravity metric for the ordering, wherein the gravity metric is computed as a sum of pairwise terms in which each pairwise term comprises a fraction with the accepted correlation coefficient of a corresponding pair of classes of the N classes in the numerator and a distance metric in the denominator that is indicative of a distance in the ordering between the classes of the corresponding pair of the classes; and
displaying a confusion matrix for a classifier to be visualized, the displayed confusion matrix having the N classes ordered in the determined ordering along an X-axis of the confusion matrix and having the N classes ordered in the determined ordering along a Y-axis of the confusion matrix, and the value of each cell of the displayed confusion matrix corresponding to match counts between the class along the X-axis class at which the cell is located and the class along the Y-axis at which the cell is located.

2. The non-transitory computer readable medium of claim 1, wherein the gravity metric comprises:
$$G = {\sum }_{i = 0}^{N - 1} {\sum }_{j = i + 1}^{N - 1} \frac{{c}_{ij}}{{r}_{{ij}^{q}}}$$
where G is the gravity metric, and cᵢⱼ is the accepted correlation coefficient for the pair of classes i and j, and rᵢⱼ is a distance metric indicative of a distance in the ordering between the classes i and j, and q is a positive real value.

3. The non-transitory computer readable medium of claim 1, wherein the gravity metric comprises:
$$G = {\sum }_{i = 0}^{N - 1} ( {\sum }_{j = i + 1}^{min ( i + {D}_{v} - 1 , N - 1 )} \frac{{c}_{ij}}{{r}_{{ij}^{q}}} )$$
where G is the gravity metric, and cᵢⱼ is the accepted correlation coefficient for the pair of classes i and j, and rᵢⱼ is a distance metric indicative of a distance in the ordering between the classes i and j, and q is a positive real value, and Dᵥ is a vanishing distance having a value between 2 and N.

4. The non-transitory computer readable medium of claim 1, wherein q=1.

5. The non-transitory computer readable medium of claim 1, wherein q>1.

6. The non-transitory computer readable medium of claim 1, wherein the determining of the ordering of the N classes that maximizes a gravity metric for the ordering includes:
computing the gravity metric for each of the N! orderings of the N classes; and
selecting the determined ordering as the ordering of the N classes for which the computed gravity metric is largest.

7. The non-transitory computer readable medium of claim 1, wherein the N classes are N clinical findings, and the classifier to be visualized is a classifier configured to classify whether each of the N clinical findings is present in an input medical image.

8. The non-transitory computer readable medium of claim 7, wherein the computing of the match counts between each pair of classes of the N classes using the classifier to be visualized includes performing operations including:
processing each medical image of a plurality of medical images using the classifier to be visualized to determine, for each image, which of the N clinical findings is present in the medical image, and
determining classifier-computed match counts based on rates of co-occurrences of pairs of clinical findings in the output of the processing.

9. The non-transitory computer readable medium of claim 1, wherein the displayed confusion matrix comprises a heat map in which the value of each cell of the displayed confusion matrix corresponding to the match counts between the class along the X-axis class at which the cell is located and the class along the Y-axis at which the cell is located is represented as a color.

10. The non-transitory computer readable medium of claim 1, wherein the classifier to be visualized comprises a single N-class classifier or N single-class classifiers.

11. A classifier visualization method, comprising:
receiving or determining an accepted correlation coefficient between each pair of classes of N classes of classes of N classes, wherein N is equal to or greater than four, wherein the N classes are N clinical findings, and a classifier to be visualized is a classifier configured to classify whether each of the N clinical findings is present in an input medical image;
determining an ordering of the N classes that maximizes a gravity metric for the ordering, wherein the gravity metric is computed as a sum of pairwise terms in which each pairwise term comprises a fraction with the accepted correlation coefficient of a corresponding pair of classes of the N classes in the numerator and a distance metric in the denominator that is indicative of a distance in the ordering between the classes of the corresponding pair of the classes;
processing each medical image of a plurality of medical images using the classifier to be visualized to determine, for each image, which of the N clinical findings is present in the medical image;
determining classifier-computed match counts based on rates of co-occurrences of pairs of clinical findings in the output of the processing; and
displaying a confusion matrix for a classifier to be visualized, the displayed confusion matrix having the N classes ordered in the determined ordering along an X-axis of the confusion matrix and having the N classes ordered in the determined ordering along a Y-axis of the confusion matrix, and the value of each cell of the displayed confusion matrix corresponding to the match counts between the class along the X-axis class at which the cell is located and the class along the Y-axis at which the cell is located.

12. The method of claim 11, wherein the gravity metric comprises:
$$G = {\sum }_{i = 0}^{N - 1} {\sum }_{j = i + 1}^{N - 1} \frac{{c}_{ij}}{{r}_{{ij}^{q}}}$$
where G is the gravity metric, and cᵢⱼ is the accepted correlation coefficient for the pair of classes i and j, and rᵢⱼ is a distance metric indicative of a distance in the ordering between the classes i and j, and q is a positive real value.

13. The method of claim 11, wherein the gravity metric comprises:
$$G = {\sum }_{i = 0}^{N - 1} ( {\sum }_{j = i + 1}^{min ( i + {D}_{v} - 1 , N - 1 )} \frac{{c}_{ij}}{{r}_{{ij}^{q}}} )$$
where G is the gravity metric, and cᵢⱼ is the accepted correlation coefficient for the pair of classes i and j, and rᵢⱼ is a distance metric indicative of a distance in the ordering between the classes i and j, and q is a positive real value, and Dᵥ is a vanishing distance having a value between 2 and N.

14. The method of claim 12, wherein q=1.

15. The method of claim 12, wherein q>1.
